(19) United States
(12) Patent Application Publication
(10) Pub. No.: US 2020/0253556 A1
(43) Pub. Date: Aug. 13, 2020

(54) BIOLOGICAL INFORMATION MEASUREMENT APPARATUS, WEARING ASSIST METHOD AND WEARING ASSIST PROGRAM

(71) Applicants: OMRON HEALTHCARE CO., LTD., Muko-shi (JP); OMRON CORPORATION, Kyoto-shi (JP)

(72) Inventors: Hiroshi NAKAJIMA, Kyoto-shi (JP); Hirotaka WADA, Kyoto-shi (JP); Tomohiro KUKITA, Kyoto (JP); Daisuke NOZAKI, Kyoto (JP); Natsuko HORIGUCHI, Kyoto (JP); Tamio UEDA, Takatsuki-shi (JP)

(73) Assignees: OMRON HEALTHCARE CO., LTD., Muko-shi (JP); OMRON CORPORATION, Kyoto-shi (JP)

(21) Appl. No.: 16/860,464

(22) Filed: Apr. 28, 2020

Related U.S. Application Data
(63) Continuation of application No. PCT/JP2018/043767, filed on Nov. 28, 2018.

(30) Foreign Application Priority Data
Dec. 4, 2017 (JP) 2017-232688

(51) Int. Cl. A61B 5/00; A61B 5/022; G06Q 10/10; G16H 40/67; G01S 19/01

(52) U.S. Cl. CPC A61B 5/6844; A61B 5/681; G01S 19/01; G06Q 10/1097; G16H 40/67; A61B 5/02233

(57) ABSTRACT

A biological information measurement apparatus includes a set time information acquisition unit configured to acquire information indicating a set time point being set to determine whether or not a subject is wearing the biological information measurement apparatus, a determination unit configured to determine whether or not the subject is wearing the biological information measurement apparatus at the set time point, and an instruction signal output unit configured to, if the determination unit determines that the subject is not wearing the biological information measurement apparatus at the set time point, output an instruction signal instructing provision of assistance to the subject in wearing the biological information measurement apparatus.

### CROSS-REFERENCE TO RELATED APPLICATIONS

This application is a Continuation Application of PCT Application No. PCT/JP2018/043767, filed Nov. 28, 2018 and based upon and claiming the benefit of priority from Japanese Patent Application No. 2017-232688, filed Dec. 4, 2017, the entire contents of all of which are incorporated herein by reference.

### FIELD

The present invention relates to a biological information measurement apparatus for which the wearing is assisted, and a wearing assist method and wearing assist program for assisting the wearing of the biological information measurement apparatus.

### BACKGROUND

In recent years, wearable blood pressure monitors that can measure blood pressure at any desired location have been developed as disclosed, for example, in Jpn. Pat. Appln. KOKAI Publication No. 2017-023546.

The utilization of blood pressure values obtained under various circumstances is expected, for example, for estimation of any problem in the body of the subject. In this regard, a wearable blood pressure monitor should be kept attached to the measurement area of the subject.

The subject, however, may temporarily remove the blood pressure monitor from the measurement area, for example, after returning home, when taking a bath, changing clothes, or doing strenuous activities. If the subject forgets to put the blood pressure monitor back on after the bath or activities, the blood pressure of the subject may not be measured under a situation where the measurement of the blood pressure is recommended, which is undesirable.

### SUMMARY

In the first aspect of the present invention, a biological information measurement apparatus includes a set time information acquisition unit configured to acquire information indicating a set time point being set to determine whether or not a subject is wearing the biological information measurement apparatus, a determination unit configured to determine whether or not the subject is wearing the biological information measurement apparatus at the set time point, and an instruction signal output unit configured to, if the determination unit determines that the subject is not wearing the biological information measurement apparatus at the set time point, output an instruction signal instructing provision of assistance to the subject in wearing the biological information measurement apparatus.

According to the first aspect of the present invention, the subject will remember the need to wear the biological information measurement apparatus. As a result, the failure to wear the biological information measurement apparatus by the subject can be reduced.

In the second aspect of the present invention, the set time point is a time point for performing measurement of biological information or a time point a predetermined time length prior to the time point for performing the measurement of the biological information, and the set time point is based on a time point entered through a user interface for entering the time point for performing the measurement of the biological information.

According to the second aspect of the present invention, the subject can reduce the failure to wear the biological information measurement apparatus at a time point when the blood pressure value should preferably be acquired. As a result, the subject can reduce the possibility of missing the chance for the blood pressure measurement at a time point when a blood pressure value is preferably acquired.

In the third aspect of the present invention, the set time point is a time point scheduled for the subject to leave a predetermined location or a time point a predetermined time length prior to the time point scheduled for the subject to leave the predetermined location, and the set time point is based on a time point entered through a user interface for entering the time point scheduled for the subject to leave the predetermined location.

According to the third aspect of the present invention, the subject can reduce the failure to wear the biological information measurement apparatus when leaving a predetermined location. As a result, the subject can reduce the number of days during which the blood pressure measurement cannot be performed between departure from a predetermined location and returning to the predetermined location.

In the fourth aspect of the present invention, the biological information measurement apparatus according to any one of the first to third aspects further includes a current location information acquisition unit configured to acquire information indicating a current location of the biological information measurement apparatus, and the instruction signal output unit changes a type of the assistance in accordance with the current location.

According to the fourth aspect of the present invention, the biological information measurement apparatus can determine the type of assistance with consideration of the people surrounding the subject in accordance with the current location of the biological information measurement apparatus. As a result, the assistance can be performed by the biological information measurement apparatus in a manner that will not annoy the people around the subject.

In the fifth aspect of the present invention, the biological information measurement apparatus according to any one of the first to third aspects further includes a priority information acquisition unit configured to acquire information indicating a priority level associated with the set time point, and wherein the instruction signal output unit determines whether output of the instruction signal is required, in accordance with the priority level.

According to the fifth aspect of the present invention, the biological information measurement apparatus may omit the process of outputting an instruction signal, depending on the priority level. As a result, the biological information measurement apparatus can reduce the processing load in relation to outputting instruction signals. In addition, the subject will not receive more assistance for wearing the biological information measurement apparatus than necessary. As a result, the subject can comfortably acquire the habit of wearing the biological information measurement apparatus.

In the sixth aspect of the present invention, the biological information measurement apparatus according to any one of the first to third aspects further includes a schedule information acquisition unit configured to acquire information indicating a schedule of the subject, and the instruction signal output unit sets a time point for providing the assistance in accordance with the schedule.

According to the sixth aspect of the present invention, the subject should not feel stress due to receiving the assistance of wearing the biological information measurement apparatus in a time slot related to a scheduled event.

In the seventh aspect of the present invention, a wearing assist method includes acquiring information indicating a set time point being set to determine whether or not a subject is wearing a biological information measurement apparatus, determining whether or not the subject is wearing the biological information measurement apparatus at the set time point, and if it is determined that the subject is not wearing the biological information measurement apparatus at the set time point, outputting an instruction signal instructing provision of assistance to the subject in wearing the biological information measurement apparatus.

According to the seventh aspect of the present invention, the wearing assist method can achieve the same advantageous effects as in the above first aspect.

In the eighth aspect of the present invention, a wearing assist program causes a computer to function as the units included in the biological information measurement apparatus according to any one of the first to sixth aspects.

According to the eighth aspect of the present invention, the wearing assist program can achieve the same advantageous effects as in the above first aspect.

According to the present invention, there is provided a technique that can reduce the subject's failure to wear a biological information measurement apparatus.

### BRIEF DESCRIPTION OF THE DRAWINGS

FIG. 1 is a diagram showing the appearance of a blood pressure monitor according to one embodiment.

FIG. 2 is a block diagram of the blood pressure monitor according to the embodiment.

FIG. 3 is a cross-sectional view of the blood pressure monitor according to the embodiment.

FIG. 4 is a functional block diagram of the blood pressure monitor according to the embodiment.

FIG. 5 is a flowchart illustrating the procedure for assisting in the attachment of the blood pressure monitor according to the embodiment.

FIG. 6 is a flowchart illustrating the procedure of changing the type of assistance in accordance with the current location of the blood pressure monitor according to the embodiment.

FIG. 7 is a flowchart illustrating the procedure for determining whether an instruction signal needs to be output in accordance with the priority level according to the embodiment.

FIG. 8 is a flowchart illustrating the procedure for setting a time point to execute the assistance in accordance with a scheduled event of the subject according to the embodiment.

### DETAILED DESCRIPTION

Embodiments of the present invention will be described below with reference to the drawings.

According to one embodiment of the present invention, there is provided a biological information measurement apparatus, a wearing assist method and a wearing assist program which can reduce the failure of wearing the apparatus by the subject.

### Embodiments

### Configuration of Blood Pressure Monitor

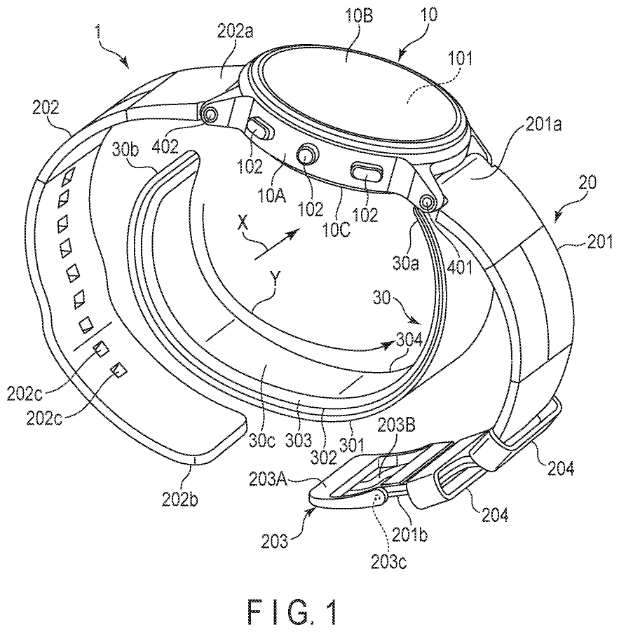

FIG. 1 is a diagram illustrating the appearance of a blood pressure monitor 1, which is an embodiment of the biological information measurement apparatus according to the present invention.

The blood pressure monitor 1 is a wristwatch-type wearable device. The blood pressure monitor 1 is provided with a blood pressure measurement function as a blood pressure measuring unit, as well as various other information processing functions. The information processing functions may include an activity measurement function, pedometer function, sleep state measurement function, and environment (temperature and humidity) measurement function. The blood pressure monitor 1 may be of a type which initiates the blood pressure measurement, based upon a start command input by the subject for the blood pressure measurement or upon a trigger signal autonomously generated by the blood pressure monitor 1. The blood pressure measurement is an example of the biological information measurement.

The blood pressure monitor 1 includes a main body 10, a belt 20, and a cuff structure 30.

The structure of the main body 10 will be described.

The main body 10 is configured to contain multiple components including the components of the control system of the blood pressure monitor 1.

The main body 10 includes a casing 10A, a watch glass 10B, and a back cover 10C.

The casing 10A may be shaped into a substantially short cylinder. The casing 10A is provided, on each of the two sides of its side surface, with a pair of protruding lugs for attaching the belt 20.

The watch glass 10B is attached to the top of the casing 10A. The watch glass 10B may be circular.

The back cover 10C is detachably attached to the bottom of the casing 10A in a manner to face the watch glass 10B.

The main body 10 is provided with a display unit 101 and an operation unit 102.

The display unit 101 displays various kinds of information. The display unit 101 is arranged in the main body 10 so as to be viewable through the watch glass 10B for the subject. The display unit 101 may be a liquid crystal display (LCD). Alternatively, the display unit 101 may be an organic electroluminescence (EL) display. The display unit 101 is not limited thereto, as long as it is provided with the function of displaying various types of information. The display unit 101 may include light emitting diodes (LED).

The operation unit 102 is a user interface for entering various commands to the blood pressure monitor 1. The operation unit 102 is provided on the side of the main body 10. The operation unit 102 may include one or more push-button switches. Alternatively, the operation unit 102 may be a touch-panel switch of a pressure-sensitive (resistive) or proximity-sensitive (capacitance) type. The operation unit 102 is not limited thereto, as long as it is provided with the function of entering various commands to the blood pressure monitor 1.

An example of the switch of the operation unit 102 will be described below.

The operation unit 102 includes a measurement switch to instruct the starting or stopping of a blood pressure measurement. The operation unit 102 may also include a home switch for returning the display screen of the display unit 101 to a predetermined home screen, and a record retrieve switch for displaying measurement records such as the previous blood pressure and activity amount on the display unit 101.

The main body 10 includes a plurality of components other than the display unit 101 and operation unit 102. These components of the main body 10 will be discussed later.

The structure of the belt 20 will be described.

The belt 20 is configured to be wound around the subject's measurement area (e.g., the left wrist). The width direction of the belt 20 will be referred to as the X direction. The direction of the belt 20 winding around the measurement area will be referred to as the Y direction.

The belt 20 includes a first belt unit 201, a second belt unit 202, a buckle 203, and a belt holder 204.

The first belt unit 201 is a band extending from the main body 10 in one direction thereof (to the right side in FIG. 1). The proximal portion 201a of the first belt unit 201 closer to the main body 10 is attached rotatably with respect to the pair of lugs of the main body 10 by way of a connecting rod 401.

The second belt unit 202 is a band extending from the main body 10 in the other direction (to the left side in FIG. 1). The proximal portion 202a of the second belt unit 202 closer to the main body 10 is rotatably attached to the pair of lugs of the main body 10 by way of a connecting rod 402. A plurality of small openings 202c are provided to penetrate the second belt unit 202 in its thickness direction between the proximal portion 202a and the distal portion 202b, which is farther from the main body 10, of the second belt unit 202.

The buckle 203 is configured to buckle the first belt unit 201 and the second belt unit 202 to each other. The buckle 203 is attached to the distal portion 201b of the first belt unit 201 that is farther from the main body 10. The buckle 203 includes a frame 203A, a bar 203B, and a connecting rod 203C.

The frame 203A and bar 203B are rotatably attached to the distal portion 201b of the first belt unit 201 by way of the connecting rod 203C. The frame 203A and bar 203B are made of, for example, a metal material. Alternatively, the frame 203A and bar 203B may be made of a plastic material. When the first belt unit 201 and second belt unit 202 are buckled to each other, the distal portion 202b of the second belt unit 202 is threaded through the frame 203A. The bar 203B is inserted into one of the small openings 202c of the second belt unit 202.

The belt holder 204 is provided between the proximal portion 201a and the distal portion 201b of the first belt unit 201. When the first belt unit 201 and second belt unit 202 are buckled to each other, the distal portion 202b of the second belt unit 202 is threaded through the belt holder 204.

The structure of the cuff structure 30 will be described.

The cuff structure 30 is configured so as to squeeze the measurement area at the time of the blood pressure measurement.

The cuff structure 30 is formed into a band extending along the Y direction. The cuff structure 30 faces the inner surface of the belt 20. The one end 30a of the cuff structure 30 is attached to the main body 10. The other end 30b of the cuff structure 30 is a free end. The cuff structure 30 therefore can be separated from the inner surface of the belt 20.

The cuff structure 30 includes a curler 301, a pressing cuff 302, a back plate 303, and a sensing cuff 304.

The curler 301 is arranged on the outermost surface of the cuff structure 30. The curler 301 is curved along the Y direction in the natural state. The curler 301 is a resin plate having a certain flexibility and rigidity. The resin plate may be made of polypropylene.

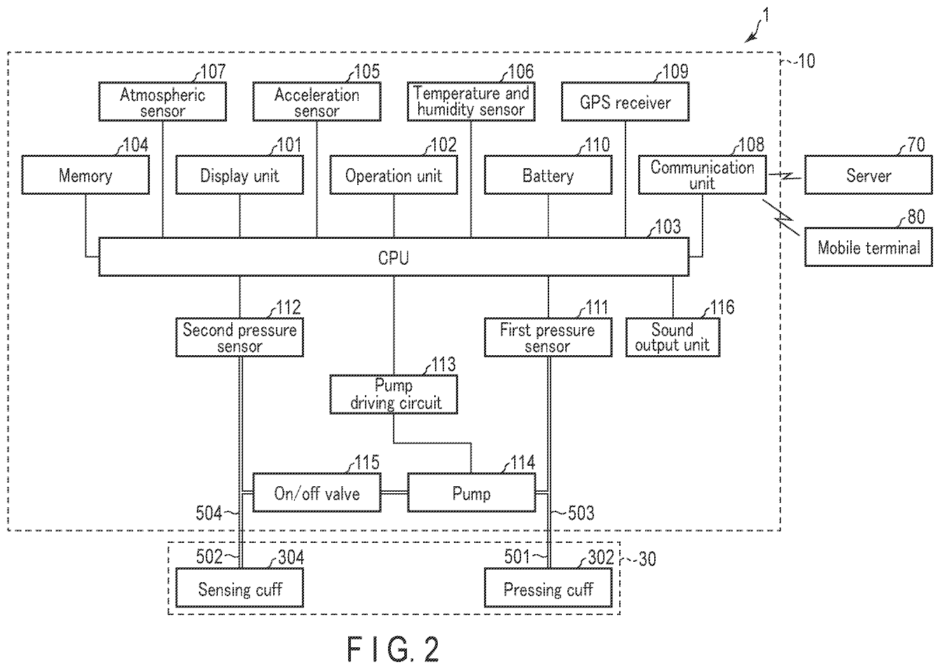

The pressing cuff 302 is arranged along the inner surface of the curler 301. The pressing cuff 302 is formed into a bag. A flexible tube 501 (shown in FIG. 2) is attached to the pressing cuff 302. The flexible tube 501 is a component for supplying a pressure transfer fluid (also referred to simply as “fluid”) from the main body 10 side to the pressing cuff 302 or to discharge the fluid from the pressing cuff 302. The fluid may be air. When the fluid is supplied to the pressing cuff 302, the pressing cuff 302 expands to squeeze the measurement area.

The pressing cuff 302 may include two fluid bags layered in the thickness direction. Each of the fluid bags may be made of an elastic polyurethane sheet. When the fluid is supplied to the pressing cuff 302, the fluid flows into the fluid bags. With the fluid bags expanded, the pressing cuff 302 is expanded.

The back plate 303 is arranged along the inner surface of the pressing cuff 302. The back plate 303 is formed into a band. The back plate 303 may be made of a resin. The resin may be polypropylene. The back plate 303 functions as a reinforcing plate. The back plate 303 therefore can transmit the pressing force from the pressing cuff 302 entirely to the sensing cuff 304.

V-shaped or U-shaped grooves in a cross sectional view extend in the X direction in the inner and outer surfaces of the back plate 303, and are arranged apart from and in parallel to each other with respect to the Y direction. The back plate 303, which is easily bent, would not obstruct the bending of the cuff structure 30.

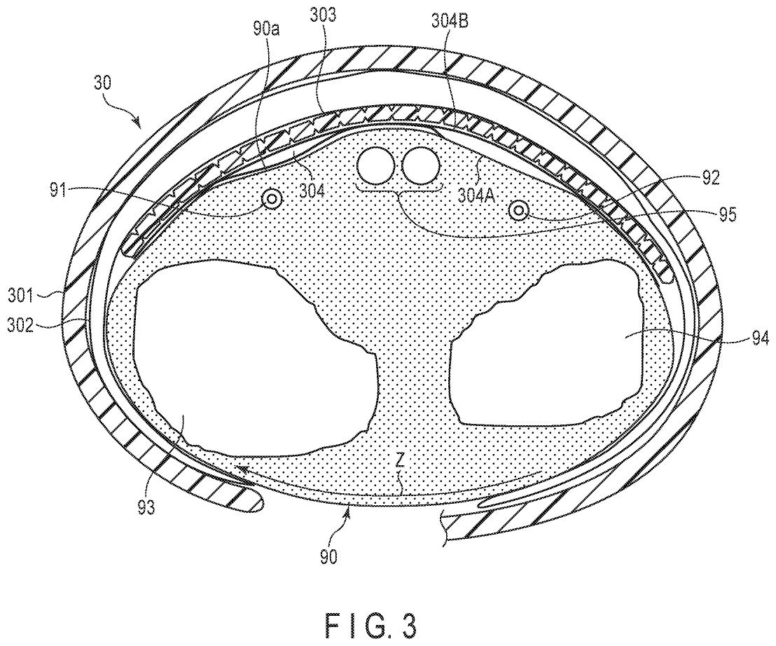

The sensing cuff 304 is arranged along the inner surface of the back plate 303. The sensing cuff 304 is formed into a bag. The sensing cuff 304 includes a first sheet 304A (shown in FIG. 3) and a second sheet 304B (shown in FIG. 3) facing the first sheet 304A. The first sheet 304A corresponds to the inner surface 30c of the cuff structure 30. This means that the first sheet 304A is brought into contact with the measurement area. The second sheet 304B faces the inner surface of the back plate 303. The first sheet 304A and second sheet 304B may be elastic polyurethane sheets. A flexible tube 502 (shown in FIG. 2) is attached to the sensing cuff 304. The flexible tube 502 is a component for supplying fluid to the sensing cuff 304 or discharging fluid from the sensing cuff 304.

Next, a plurality of components arranged in the main body 10 will be described.

FIG. 2 is a block diagram illustrating the hardware structure of the blood pressure monitor 1.

The main body 10 includes, in addition to the above-mentioned display unit 101 and operation unit 102, a central processing unit (CPU) 103, a memory 104, an acceleration sensor 105, a temperature and humidity sensor 106, an atmospheric sensor 107, a communication unit 108, a global positioning system (GPS) receiver 109, a battery 110, a first pressure sensor ill, a second pressure sensor 112, a pump driving circuit 113, a pump 114, an on/off valve 115, and a sound output unit 116.

The CPU 103 is an example of a processor that forms a computer. The CPU 103, which serves as a controller, performs various functions in accordance with the programs stored in the memory 104, and controls the operations of the components of the blood pressure monitor 1. The configurations of the components provided in the CPU 103 will be described later.

The memory 104 stores programs that cause the CPU 103 to function as the respective components of the blood pressure monitor 1. The program may also be regarded as instructions to have the CPU 103 operate. The memory 104 further stores the data used for controlling the blood pressure monitor 1, the setting data for setting the functions of the blood pressure monitor 1, and the data of the measurement results of blood pressure values. The memory 104 is also used as a work memory when a program is implemented.

The memory 104 further stores information indicating one or more set time points. A set time point represents the time at which the blood pressure monitor 1 determines whether or not the subject is wearing the blood pressure monitor 1. The set time point can be suitably set by the subject.

For example, the set time point may be the time at which the blood pressure monitor 1 autonomously performs a blood pressure measurement. The time for the blood pressure measurement is when the blood pressure value of the subject should preferably be obtained. The time for the blood pressure measurement may be, but is not particularly limited to, a time point presumably before going to bed (e.g., 11 p.m.), a time point presumably after getting up (e.g., 7 a.m.), and a time point while at a workplace or school (e.g., 2 p.m.). The memory 104 may store one or more time points at which the blood pressure measurement is to be performed.

The set time point may be a point in time a first predetermined time length prior to the time point of the blood pressure monitor 1 autonomously performing the blood pressure measurement. The first predetermined time length may be determined in consideration of a time length required for the subject to wear the blood pressure monitor 1. The first predetermined time length may be, but is not limited to, 5 minutes. This will allow the subject to wear the blood pressure monitor 1 prior to the time point at which the blood pressure monitor 1 performs the autonomous blood pressure measurement.

The set time point in this example is based on the time point entered by the subject on the operation unit 102. The operation unit 102 is a component for entering a time point at which the blood pressure monitor 1 performs an autonomous blood pressure measurement. The set time point may be a time point entered by the subject on the operation unit 102, or a time point the first predetermined time length prior to the time point entered by the subject on the operation unit 102.

In another example, the set time point is a time point at which the subject is scheduled to leave a predetermined location. The predetermined location may be home. In this example, the time point when the subject leaves the predetermined location may be, but is not particularly limited to, the time scheduled for leaving home for work or for school. In another example, the predetermined location may be a workplace or school. If this is the case, the time when the subject leaves the predetermined location may be, but is not particularly limited to, the time scheduled for leaving the workplace after work, or the time scheduled for leaving school after being dismissed. The memory 104 may store one or more time points at which the subject is scheduled to leave a predetermined location.

The set time point may be a time point a second predetermined time length prior to the time when the subject is scheduled to leave the predetermined location. The second predetermined time length may be set in consideration of the possibility of the subject leaving the predetermined location earlier than usual. The second predetermined time length may be, but is not limited to, 10 minutes. This will prevent the subject from failing to wear the blood pressure monitor 1 even if the subject needs to leave the predetermined location earlier than usual.

The set time point in this example is based on the time point entered by the subject on the operation unit 102. The operation unit 102 is a component for entering a time point at which the subject is scheduled to leave a predetermined location. The set time point may be a time point entered by the subject on the operation unit 102 or a time point the second predetermined time length prior to the time point entered by the subject on the operation unit 102.

The memory 104 may store the above-mentioned one or more time points to perform blood pressure measurement and/or one or more time points at which the subject is scheduled to leave a predetermined location.

The memory 104 stores the set time point in association with a priority level. For instance, the set time point may be associated with either one of a first priority level or a second priority level lower than the first priority level. The subject may designate either the first priority level or second priority level for the set time point, in accordance with the degree to which the assistance for wearing the blood pressure monitor 1 is required. In this example, the set time point associated with either one of the two different priority levels is explained, but it is not particularly limited. The set time point may be associated with one of three or more priority levels.

For example, the priority level for the time point presumably before bedtime and after getting up may be the first priority level, since the blood pressure values of the time presumably before bedtime and after getting up provide useful information for estimating a problem in the subject's body. Alternatively, the priority level for the time point presumably before bedtime and after getting up may be the second priority level. This is because the subject may be asleep and the assistance by at least one of the blood pressure monitor 1 and the mobile terminal 80 may wake the subject up.

For example, the priority of the time point at which the subject is scheduled to leave the predetermined location or the priority of a time point the second predetermined time length prior to the time point the subject is scheduled to leave the predetermined location may be the first priority level, since the blood pressure cannot be measured after the subject leaves the predetermined location until the subject returns to the predetermined location.

For example, the priority of a time point during the stay at a workplace or school may be the first priority level. If the subject is suspected of so-called workplace hypertension, the need to measure the blood pressure during the stay at a workplace or school is increased. Alternatively, the priority of the time point presumably during the stay at a workplace or school may be the second priority level. For a subject who is not suspected of so-called workplace hypertension, the need to measure blood pressure during the stay at the workplace or school will be low.

The acceleration sensor 105 is a three-axis acceleration sensor. The acceleration sensor 105 outputs to the CPU 103 an acceleration signal representing the acceleration in three directions orthogonal to each other. Through the use of the acceleration signal, the CPU 103 can calculate the amount for various activities including not only the subject's walking activity but also housework and desk work activities. The amount of activity may be an index relating to the activity of the subject, such as a moving (walking) distance, calorie expenditure, or amount of fat burned. Through the use of the acceleration signal, the CPU 103 can also estimate the subject's sleep state by detecting the roll-over state.

The temperature and humidity sensor 106 measures the ambient temperature and humidity in the surroundings of the blood pressure monitor 1. The temperature and humidity sensor 106 outputs ambient data representing the ambient temperature and humidity to the CPU 103. The CPU 103 stores the ambient data in the memory 104 in association with the measurement time at the temperature and humidity sensor 106. The air temperature (change in air temperature) can be considered to be one of the factors for triggering variance in human blood pressure. The ambient data is therefore information indicating a determinant of the variance in blood pressure of the subject.

The atmospheric sensor 107 detects atmospheric pressure. The atmospheric sensor 107 outputs atmospheric pressure data to the CPU 103. The CPU 103 is configured to measure the number of steps walked, the number of steps briskly walked, and the number of stair-ascending steps by the subject, using the atmospheric pressure data and acceleration signals.

The communication unit 108 is an interface for connecting the blood pressure monitor 1 to at least one of a server 70 and mobile terminal 80. The mobile terminal 80 may be a smartphone or a tablet terminal. This mobile terminal 80 belongs to the subject. The communication unit 108 is controlled by the CPU 103. The communication unit 108 transmits information to at least one of the server 70 and the mobile terminal 80 via a network. The communication unit 108 passes to the CPU 103 the information received from at least one of the server 70 and mobile terminal 80 via the network. The communication over this network may be performed in a wireless or wired manner. The network may be the Internet, but is not limited thereto. The network may be of any other type such as an intra-hospital local area network (LAN), or one-to-one communication may be performed using a USB cable or the like. The communication unit 108 may include a micro-USB connector. The communication unit 108 may transmit information to the mobile terminal 80 by short range communication such as Bluetooth (registered trademark).

The GPS receiver 109 receives GPS signals transmitted from a plurality of GPS satellites, and outputs the received GPS signals to the CPU 103. The CPU 103 calculates the current location of the blood pressure monitor 1, or in other words the current location of the subject wearing the blood pressure monitor 1, by carrying out a distance measurement based on the GPS signals. The blood pressure monitor 1 may not necessarily include this distance measurement function realized by the GPS receiver 109 and the CPU 103. If this is the case, the blood pressure monitor 1 acquires the information indicating the current location calculated by the mobile terminal 80, from the mobile terminal 80 via the communication unit 108. The current location calculated by the mobile terminal 80 corresponds to the current location of the blood pressure monitor 1.

The battery 110 may be a rechargeable secondary battery. The battery 110 supplies power to the components provided in the main body 10. The battery 110 may supply power to the display unit 101, operation unit 102, CPU 103, memory 104, acceleration sensor 105, temperature and humidity sensor 106, atmospheric sensor 107, communication unit 108, GPS receiver 109, first pressure sensor 111, second pressure sensor 112, pump driving circuit 113, pump 114, on/off valve 115 and sound output unit 116.

The first pressure sensor 111 may be a piezoresistive pressure sensor. The first pressure sensor 111 detects the pressure inside the pressing cuff 302 through the flexible tube 501 and first channel member 503 that form the first channel. The first pressure sensor 111 outputs the pressure data to the CPU 103.

The second pressure sensor 112 may be a piezoresistive pressure sensor. The second pressure sensor 112 detects the pressure inside the sensing cuff 304 through the flexible tube 502 and second channel member 504 that form the second channel. The second pressure sensor 112 outputs the pressure data to the CPU 103.

The pump driving circuit 113 drives the pump 114 on the basis of the control signal from the CPU 103.

The pump 114 may be a piezoelectric pump. The pump 114 is connected to the pressing cuff 302 by way of the first channel in a manner such that the fluid can flow through. The pump 114 can supply the fluid to the pressing cuff 302 through the first channel. The pump 114 is provided with an exhaust valve (not shown) the opening and closing of which is controlled in accordance with the on/off state of the pump 114. That is, the exhaust valve closes when the pump 114 is turned on, allowing the fluid to fill the pressing cuff 302. On the other hand, when the pump 114 is turned off, the exhaust valve opens, releasing the fluid in the pressing cuff 302 to the atmosphere through the first channel. The exhaust valve has a check valve function so that the fluid being released will not flow backward.

The pump 114 is also connected to the sensing cuff 304 by way of the second channel in a manner such that the fluid can flow through. The pump 114 can supply the fluid to the sensing cuff 304 through the second channel.

The on/off valve 115 is provided in the middle of the second channel member 504. The on/off valve 115 may be a normally open solenoid valve. The opening and closing (degree of opening) of the on/off valve 115 is controlled based on the control signal from the CPU 103. When the on/off valve 115 is in the open state, the pump 114 can supply fluid to the sensing cuff 304 through the second channel.

Next, the state of the blood pressure monitor 1 being attached to the measurement area (also referred to as the “attached state”) will be explained.

FIG. 3 is a diagram illustrating a vertical section of the left wrist 90 that is the measurement area in the attached state. The main body 10 and the belt 20 are omitted from this drawing. The left wrist 90 in FIG. 3 includes a radial artery 91, ulnar artery 92, radius 93, ulna 94 and tendon 95.

In this state, the curler 301 extends along the circumference (Z direction) of the left wrist 90. The pressing cuff 302 extends along the Z direction on the inner circumferential side of the curler 301. The back plate 303 is interposed between the pressing cuff 302 and the sensing cuff 304, and extends along the Z direction. The sensing cuff 304 is in contact with the left wrist 90 and extends in the Z direction in such a manner as to cross the arterial passage area 90a of the left wrist 90. The belt 20, curler 301, pressing cuff 302 and back plate 303 serve as a pressing member capable of producing a pressing force toward the left wrist 90, and squeeze the left wrist 90 by way of the sensing cuff 304.

Next, the configuration of the components realized by the CPU 103 will be described.

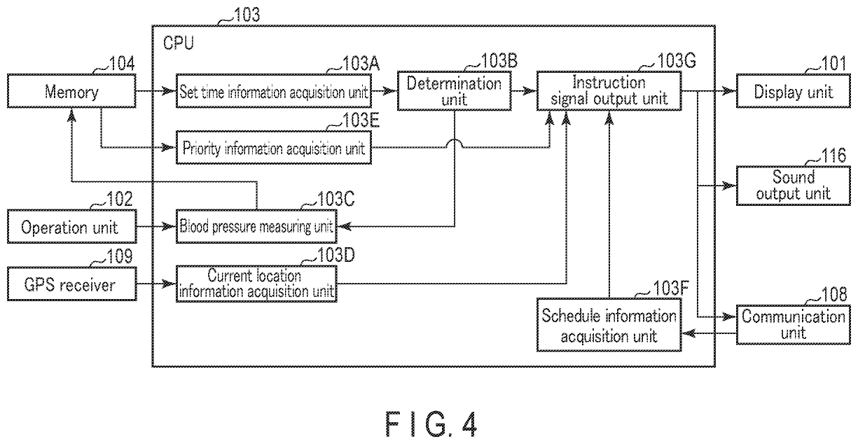

FIG. 4 is a functional block diagram of the blood pressure monitor 1. The CPU 103 implements a set time information acquisition unit 103A, a determination unit 103B, a blood pressure measuring unit 103C, a current location information acquisition unit 103D, a priority information acquisition unit 103E, a schedule information acquisition unit 103F, and an instruction signal output unit 103G. These units may be distributed over two or more processors.

The configuration of the set time information acquisition unit 103A will be described.

The set time information acquisition unit 103A acquires information indicating the set time point from the memory 104. For example, the set time information acquisition unit 103A sequentially acquires from the memory 104 the information indicating a set time point within a predetermined time length after the current time. The set time information acquisition unit 103A may acquire the information indicating the current time through the clock function of the blood pressure monitor 1. The set time information acquisition unit 103A outputs the information indicating a set time point to the determination unit 103B.

The configuration of the determination unit 103B will be described.

The determination unit 103B compares the current time with the set time point and stays on standby until the current time reaches the set time point. The determination unit 103B may acquire the information indicating the current time through the clock function of the blood pressure monitor 1. When it is determined that the current time has reached the set time point, the determination unit 103B determines whether or not the blood pressure monitor 1 is attached at the set time point. The determination unit 103B may determine whether or not the blood pressure monitor 1 is attached at the set time point in the manner indicated below. The determination unit 103B controls the pressing cuff 302 to pressurize by supplying fluid thereto, and monitors the pressure of the pressing cuff 302. When the subject is wearing the blood pressure monitor 1, the pressing cuff 302 comes into contact with the measurement area. Even if the same volume of fluid is supplied in the pressing cuff 302, the pressure of the pressing cuff 302 differs depending on whether the subject is wearing the blood pressure monitor 1. By referring to the pressure of the pressing cuff 302, the determination unit 103B can determine whether or not the blood pressure monitor 1 is attached at the set time point.

When it is determined that the subject is wearing the blood pressure monitor 1 at the set time point, the determination unit 103B may perform the process below.

If the set time point is the time for performing the blood pressure measurement or a time point the first predetermined time length prior to the blood pressure measurement, the determination unit 103B outputs to the blood pressure measuring unit 103C a signal (hereinafter also referred to as a “blood pressure measurement initiation signal”) for the blood pressure monitor 1 to autonomously perform the blood pressure measurement at the time scheduled for the blood pressure measurement.

If the set time point is the time for leaving the predetermined location or a time point the second predetermined time length prior to the time for leaving the predetermined location, the determination unit 103B terminates the process. This is because, when it is time for the subject to leave the predetermined location or it is at a time point the second predetermined time length prior to the time for leaving the predetermined location, the blood pressure monitor 1 does not need to perform blood pressure measurement.

When it is determined that the subject is not wearing the blood pressure monitor 1 at the set time point, the determination unit 103B outputs to the instruction signal output unit 103G a signal indicating that the subject is not wearing the blood pressure monitor 1.

The configuration of the blood pressure measuring unit 103C will be explained.

The blood pressure measuring unit 103C may control the blood pressure measurement of the subject in the following manner.

The blood pressure measuring unit 103C initializes the memory area of the memory 104 for the processing upon the detection of the measurement switch pressed by the subject or the detection of the above-mentioned blood pressure measurement initiation signal which triggers the blood pressure measurement to start. The blood pressure measuring unit 103C performs control so as to release the fluid from the pressing cuff 302 and sensing cuff 304 by turning the pump 114 off by way of the pump driving circuit 113, opening the exhaust valve included in the pump 114, and keeping the on/off valve 115 in an open state. The blood pressure measuring unit 103C performs control so that the adjustment of the first pressure sensor 111 and second pressure sensor 112 to 0 mmHg can be achieved. The blood pressure measuring unit 103C performs control so as to initiate the pressurization of the pressing cuff 302 and sensing cuff 304 by turning the pump 114 on by way of the pump driving circuit 113, and keeping the on/off valve 115 in an open state. The blood pressure measuring unit 103C performs control to turn the pump 114 on by way of the pump driving circuit 113, while monitoring the pressures of the pressing cuff 302 and sensing cuff 304 through the first pressure sensor 111 and second pressure sensor 112. The blood pressure measuring unit 103C performs control so as to supply fluid to the pressing cuff 302 through the first channel and to the sensing cuff 304 through the second channel. The blood pressure measuring unit 103C waits for the pressure of the sensing cuff 304 to reach a predetermined pressure (e.g., 15 mmHg) or for the driving time of the pump 114 to elapse a predetermined time length (e.g., 3 seconds). With the on/off valve 115 turned to a closed state, the blood pressure measuring unit 103C continues the control for supplying the fluid from the pump 114 to the pressing cuff 302 through the first channel. In this manner, the pressure is gradually applied to the pressing cuff 302, gradually pressurizing the left wrist 90. The back plate 303 conveys the pressing force from the pressing cuff 302 to the sensing cuff 304. As a result, the sensing cuff 304 squeezes the left wrist 90 (including the arterial passage area 90a). In this pressurization process, the blood pressure measuring unit 103C monitors the pressure Pc of the sensing cuff 304, or in other words, the pressure on the arterial passage area 90a of the left wrist 90, through the second pressure sensor 112 and acquires a pulse signal Pm as a variance component in order to calculate the blood pressure values (systolic blood pressure SBP and diastolic blood pressure DBP). Based on this pulse signal Pm, the blood pressure measuring unit 103C calculates a blood pressure values by an oscillometric method with a known algorithm applied. After calculating the blood pressure values, the blood pressure measuring unit 103C performs control so as to discharge the fluid from the pressing cuff 302 and the sensing cuff 304 by turning the pump 114 off and opening the on/off valve 115.

Under the above control, the blood pressure measuring unit 103C measures the blood pressure of the subject. The blood pressure measuring unit 103C stores the blood pressure values in the memory 104 in association with the date and time of the blood pressure measurement. The configuration of the current location information acquisition unit 103D will be described.

The current location information acquisition unit 103D acquires information indicating the current location of the blood pressure monitor 1, as described below. In one example, the current location information acquisition unit 103D receives GPS signals from the GPS receiver 109. Next, the current location information acquisition unit 103D calculates the current location of the blood pressure monitor 1 based on the GPS signals, thereby obtaining information indicating the current location of the blood pressure monitor 1. In another example, the current location information acquisition unit 103D may obtain information indicating the current location of the mobile terminal 80, from the mobile terminal 80 via the communication unit 108. The current location of the mobile terminal 80 corresponds to the current location of the blood pressure monitor 1.

The current location information acquisition unit 103D outputs the information indicating the current location of the blood pressure monitor 1 to the instruction signal output unit 103G.

The configuration of the priority information acquisition unit 103E will be described.

The priority information acquisition unit 103E obtains information indicating a priority level associated with the set time point, as described below. By referring to the set time point, the priority information acquisition unit 103E acquires information indicating a priority level associated with the set time point from the memory 104. The priority information acquisition unit 103E outputs the information indicating the priority level associated with set time point to the instruction signal output unit 103G.

The configuration of the schedule information acquisition unit 103F will be described.

The schedule information acquisition unit 103F acquires information indicating the schedule of the subject from at least one of the server 70 and the mobile terminal 80, as described below. The schedule information contains information of time slots associated with at least one or more events.

In one example, the schedule information acquisition unit 103F may acquire the schedule information of the subject from the server 70 via the communication unit 108. The schedule information of the subject is based on the scheduled event entered by the subject in the workplace, using a schedule management application. For example, the subject may use the schedule management application to enter a scheduled meeting and a related time slot. The subject can also enter scheduled events other than meetings.

In this example, information indicating the schedule of the subject is generated by the server 70. For example, the server 70 may analyze the details of the events through text mining and thereby generate the schedule information. In place of the text mining, the server 70 may refer to pull-down options used for entering a scheduled event when analyzing the details of the event and generating the schedule information. The server 70 may adopt any other method to analyze the content of events and generate the schedule information.

In another example, the schedule information acquisition unit 103F may obtain the schedule information of the subject from the mobile terminal 80. The schedule information of the subject is based on the details of the scheduled events entered by the subject using the schedule management application on the mobile terminal 80. The subject may enter private appointments, sleep schedule, and their related time slots. The subject may also enter appointments other than private appointments and sleep schedule.

In the above example, the information indicating the schedule of the subject is generated by the mobile terminal 80. The mobile terminal 80 may adopt the same method as used by the server 70 to analyze the details of events and generate the schedule information.

The configuration of the instruction signal output unit 103G will be described.

When the determination unit 103B determines that the subject is not wearing the blood pressure monitor 1 at the set time point, the instruction signal output unit 103G outputs an instruction signal instructing the provision of assistance to the subject in putting on the blood pressure monitor 1. The instruction signal output unit 103G may output an instruction signal in the following manner.

The instruction signal output unit 103G may output an instruction signal to the display unit 101. Based on the instruction signal, the display unit 101 displays an image that urges the subject to put on the blood pressure monitor 1. The image should be sufficient as long as it can make the subject aware of the need to wear the blood pressure monitor 1, and is not particularly limited.

The instruction signal output unit 103G may output an instruction signal to the sound output unit 116. Based on the instruction signal, the sound output unit 116 outputs a sound that urges the subject to put on the blood pressure monitor 1. The sound should be sufficient as long as it can make the subject aware of the need to put on the blood pressure monitor 1, and is not particularly limited. The sound output unit 116 may output an alarm, or may output a message in place of an alarm.

The instruction signal output unit 103G outputs an instruction signal to the server 70 via the communication unit 108. Based on the instruction signal, the server 70 sends an e-mail to the mobile terminal 80. The e-mail should be sufficient, as described above, as long as it can make the subject aware of the need to put on the blood pressure monitor 1, and is not particularly limited. The mobile terminal 80 displays an image of the e-mail received from the server 70.

The instruction signal output unit 103G may output an instruction signal to the mobile terminal 80 via the communication unit 108. Based on the instruction signal, the mobile terminal 80 outputs at least one of a sound and an image that urges the subject to put on the blood pressure monitor. As described above, the sound and the image should be sufficient as long as they can make the subject aware of the need to put on the blood pressure monitor 1, and are not particularly limited.

The instruction signal output unit 103G may output the instruction signal, for example, to at least one of the display unit 101, sound output unit 116, server 70, and mobile terminal 80. With the assistance of these components, the subject becomes aware of the need to put on the blood pressure monitor 1 so that the blood pressure monitor 1 can be attached.

The instruction signal output unit 103G may refer to the information of the current location of the blood pressure monitor 1 received from the current location information acquisition unit 103D and change the type of assistance, depending on the current location of the blood pressure monitor 1. Examples of changing the types of the assistance will be described later.

In addition, the instruction signal output unit 103G may refer to the information received from the priority information acquisition unit 103E regarding the priority level associated with the set time point, and thereby determine whether or not an instruction signal is required, depending on the priority level associated with the set time point. An example of determining whether an instruction signal is required will be described later.

The instruction signal output unit 103G may refer to the schedule information of the subject received from the schedule information acquisition unit 103F, and set the time for providing the assistance in accordance with the schedule of the subject. An example of setting the time point for providing the assistance will be described later.

(Operation)

The assistance provided by the blood pressure monitor 1 for the subject in wearing the blood pressure monitor 1 will be explained.

FIG. 5 is a flowchart showing the procedure of the process for assisting in the attachment of the blood pressure monitor 1 and the steps of this process.

The set time information acquisition unit 103A acquires information indicating the set time point stored in the memory 104 (step S101). At step S101, the set time information acquisition unit 103A may acquire, as the information of the set time point, information indicating at least one of the time when the blood pressure measurement is to be performed and the time when the subject is scheduled to leave a predetermined location. The set time information acquisition unit 103A may acquire, as the information of the set time point, information indicating a time point the first predetermined time length prior to the time for the blood pressure measurement. The set time information acquisition unit 103A may acquire, as the information of the set time point, information indicating a time point the second predetermined time length prior to the time when the subject is scheduled to leave the predetermined location.

The determination unit 103B determines whether or not the current time has reached the set time point (step S102). If the determination unit 103B determines that the current time has not reached the set time point (no at step S102), the determination unit 103B stays on standby until the current time reaches the set time point.

When the determination unit 103B determines that the current time has reached the set time point (yes at step S102), the determination unit 103B determines whether or not the subject is wearing the blood pressure monitor 1 at the set time point (step S103). At step S103, the determination unit 103B may monitor the pressure of the pressing cuff 302 as described above and thereby determines whether or not the subject is wearing the blood pressure monitor 1.

If the determination unit 103B determines that the subject is wearing the blood pressure monitor 1 at the set time point (yes at step S103), the blood pressure measuring unit 103C performs the blood pressure measurement on the subject (step S104). At step 5104, the blood pressure measuring unit 103C may control the respective components of the blood pressure monitor 1 in the above mentioned manner so as to control the blood pressure measurement of the subject.

If the determination unit 103B determines that the subject is not wearing the blood pressure monitor 1 at the set time point (no at step S103), the instruction signal output unit 103G outputs an instruction signal (step S105). At step S105, the instruction signal output unit 103G may output the instruction signal to at least one of the display unit 101, the sound output unit 116, the server 70, and the mobile terminal 80.

If the information of the set time point acquired by the set time information acquisition unit 103A at step S101 indicates the time when the subject is scheduled to leave a predetermined location or a time point the second predetermined time length prior to the time to leave the predetermined location, the blood pressure monitor 1 skips the operation of step S104 and terminates the process.

Next, some examples of the operation at the above described step S105 will be explained.

In one example, the instruction signal output unit 103G may change the type of the assistance, depending on the current location of the blood pressure monitor 1. In this example, the memory 104 stores location information of various locations. These locations are classified into a first location or a second location.

The first location is a place where an image output is preferable to a sound output as the assistance to the subject in wearing the blood pressure monitor 1. Examples of the first location include a workplace. When the subject is in the workplace, a sound output from the blood pressure monitor 1 or mobile terminal 80 would bother or considerably affect the surrounding people.

The second location differs from the first location. The second location is a place where a sound output is preferable to an image output as the assistance to the subject in wearing the blood pressure monitor 1. Examples of the second location include home. When the subject is at home, a sound output from the blood pressure monitor 1 or mobile terminal 80 would not largely affect the surrounding people, unlike in a workplace. In addition, the subject can more easily recognize that he/she needs to wear the blood pressure monitor 1 with a sound output than an image output by the blood pressure monitor 1 or mobile terminal 80.

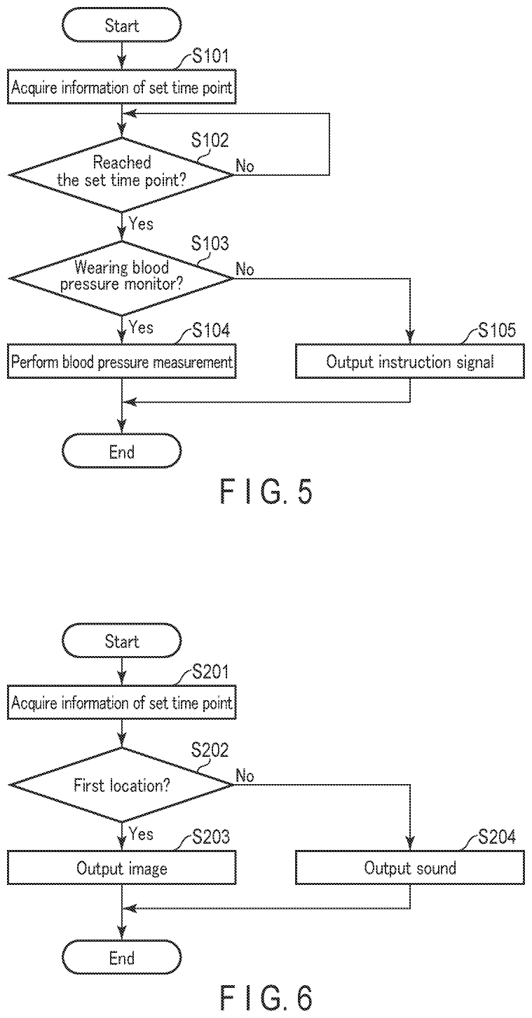

FIG. 6 is a flowchart illustrating the procedure of changing the type of the assistance in accordance with the current location of the blood pressure monitor 1 and the steps of this procedure.

The instruction signal output unit 103G acquires information indicating the current location of the blood pressure monitor 1 (step S201). At step S201, the instruction signal output unit 103G acquires the information indicating the current location of the blood pressure monitor 1 from the current location information acquisition unit 103D.

The instruction signal output unit 103G determines whether or not the current location is the first location (step S202). For instance, at step S202, the instruction signal output unit 103G may compare the information indicating the current location with the location information of various locations stored in the memory 104, and determines whether or not the current location is the first location.

If the current location is the first location (yes at step S202), the instruction signal output unit 103G outputs an instruction signal instructing the output of an image (step S203). At step S203, the instruction signal output unit 103G may output an instruction signal instructing the output of an image, to at least one of the display unit 101, the server 70, and the mobile terminal 80. The subject may recognize that the blood pressure monitor 1 needs to be attached by viewing an image displayed on at least one of the blood pressure monitor 1 and the mobile terminal 80.

If the current location is not the first location (no at step S202), the instruction signal output unit 103G outputs an instruction signal instructing the output of a sound (step S204). If the current location is not the first location, the current location is the second location. At step S204, the instruction signal output unit 103G outputs an instruction signal instructing the output of a sound to at least one of the sound output unit 116 and the mobile terminal 80. Upon hearing the sound output from at least one of the blood pressure monitor 1 and mobile terminal 80, the subject may recognize that the blood pressure monitor 1 needs to be attached.

As described above, the instruction signal output unit 103G may change the type of assistance between an image output or a sound output in accordance with the current location.

At step S204, the instruction signal output unit 103G may output an instruction signal instructing the output of an image, together with an instruction signal instructing the output of a sound. If this is the case, the subject can further reliably recognize that the blood pressure monitor 1 needs to be attached by checking both the sound and image output from at least one of the blood pressure monitor 1 and the mobile terminal 80.

In another example, the instruction signal output unit 103G may determine whether or not an instruction signal is required, depending on the priority level associated with the set time point.

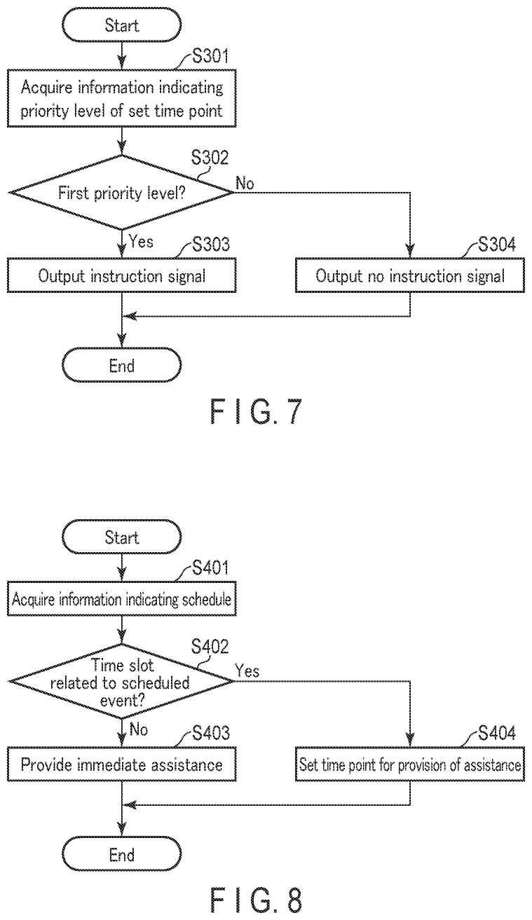

FIG. 7 is a flowchart showing the procedure for determining whether or not an instruction signal is required in accordance with the priority level associated with the set time point and the steps of this procedure.

The instruction signal output unit 103G acquires information indicating the priority level associated with the set time point from the priority information acquisition unit 103E (step S301).

The instruction signal output unit 103G determines whether or not the first priority level is associated with the set time point (step S302).

If the first priority level is associated with the set time point (yes at step S302), the instruction signal output unit 103G outputs an instruction signal (step S303). In other words, at step S303, the instruction signal output unit 103G determines that the output of an instruction signal is required.

If the first priority level is not associated with the set time point (no at step S302), the instruction signal output unit 103G does not output an instruction signal (step S304). If the first priority level is not associated with the set time point, the set time point is associated with the second priority level. In other words, at step S304, the instruction signal output unit 103G determines that the output of an instruction signal is not required.

As described above, the instruction signal output unit 103G can determine whether or not the output of the instruction signal is required, depending on whether the priority level associated with the set time point is the first priority level or the second priority level.

In another example, the instruction signal output unit 103G can set the time point at which the assistance is performed in accordance with the schedule of the subject.

FIG. 8 is a flowchart showing the procedure for setting a time point for providing the assistance in accordance with the schedule of the subject and the steps of this procedure.

The instruction signal output unit 103G acquires information indicating the schedule of the subject from the schedule information acquisition unit 103F (step S401). The schedule information contains information of time slots associated with at least one or more scheduled events.

The instruction signal output unit 103G determines whether or not the current time is included in the time slot related to a scheduled event of the subject (step S402). At step S402, the instruction signal output unit 103G can acquire the information of the current time through the clock function of the blood pressure monitor 1. If the current time is not included in the time slot related to the scheduled event (no at step S402), the instruction signal output unit 103G outputs an instruction signal to immediately provide the assistance (step S403). At step S403, the instruction signal output unit 103G may output an instruction signal that includes a setting for immediately providing the assistance, to at least one of the display unit 101, the sound output unit 116, the server 70, and the mobile terminal 80. This allows at least one of the blood pressure monitor 1 and mobile terminal 80 to immediately output at least one of an image and sound to urge the attachment of the blood pressure monitor 1.

If the current time is included in the time slot related to the scheduled event (yes at step S402), the instruction signal output unit 103G sets the provision of the assistance to a time point after the time slot related to the scheduled event (step S404).

At step S404, for example, the instruction signal output unit 103G outputs an instruction signal for providing immediate assistance, to at least one of the display unit 101, the sound output unit 116, the server 70, and the mobile terminal 80 after the time slot associated with the scheduled event. This allows at least one of the blood pressure monitor 1 and mobile terminal 80 to output at least one of an image and sound urging the attachment of the blood pressure monitor 1 after the time slot associated with the scheduled event passes. Alternatively, the instruction signal output unit 103G may immediately output an instruction signal including setting where the time point for providing the assistance is set to a time point after the time slot associated with the scheduled event passes, to at least one of the display unit 101, the sound output unit 116, the server 70, and the mobile terminal 80. This allows at least one of the blood pressure monitor 1 and mobile terminal 80 to output at least one of an image and sound to urge the attachment of the blood pressure monitor 1 after the time slot associated with the scheduled event passes.

The instruction signal output unit 103G may adjust an instruction signal by using two or more of the current location of the blood pressure monitor 1, the priority level associated with the set time point, and the schedule of the subject.

### Advantageous Effects

As discussed above in detail, the blood pressure monitor 1 according to one embodiment of the present invention is configured to output an instruction signal instructing the provision of the assistance to the subject in wearing the blood pressure monitor 1 if the subject is not wearing the blood pressure monitor 1 at the set time point. This allows the subject to recognize that the blood pressure monitor 1 needs to be attached. As a result, the failure to attach the blood pressure monitor 1 by the subject will be reduced.

Moreover, the blood pressure monitor 1 according to one embodiment of the present invention may adopt, as a set time point, the time for performing the blood pressure measurement or a time point a first predetermined time length prior to the time for performing the blood pressure measurement. The subject can thereby reduce the failure to wear the blood pressure monitor 1 at a time point when the blood pressure value should preferably be acquired. As a result, the subject can reduce the possibility of missing the chance for the blood pressure measurement at the time point when the blood pressure value should preferably be acquired.

In addition, the blood pressure monitor 1 according to one embodiment of the present invention may adopt as a set time point the time point at which the subject is scheduled to leave a predetermined location or a time point the second predetermined time length prior to the time point at which the subject is scheduled to leave the predetermined location. In this manner, the subject can reduce the failure to wear the blood pressure monitor 1 when leaving the predetermined location. As a result, the subject can reduce the number of days between leaving a predetermined location and returning to the predetermined location, during which the blood pressure cannot be measured.

Furthermore, the blood pressure monitor 1 according to one embodiment of the present invention can change the type of assistance, depending on the current location of the blood pressure monitor 1. The blood pressure monitor 1 therefore can determine the type of assistance with consideration of the people surrounding the subject, in accordance with the current location of the blood pressure monitor 1. As a result, the assistance provided by the blood pressure monitor 1 will not bother anyone surrounding the subject.

In addition, according to one embodiment of the present invention, the blood pressure monitor 1 can determine whether or not the output of an instruction signal is required, depending on the priority level associated with the set time point. The blood pressure monitor 1 can thereby omit the output of an instruction signal depending on the priority level. As a result, the blood pressure monitor 1 can reduce the load of processing in connection with the output of an instruction signal. In addition, the subject will not receive assistance by the blood pressure monitor 1 beyond necessity. As a result, the subject can pleasantly become accustomed to wearing the blood pressure monitor 1.

Moreover, the blood pressure monitor 1 according to one embodiment of the present invention can set the time for providing the assistance in accordance with the schedule of the subject. In this manner, the subject will not feel stress due to receiving the assistance of wearing the blood pressure monitor 1 in a time slot associated with a scheduled event.

### Other Embodiments

The blood pressure monitor 1 is not limited to a blood pressure monitor of a type configured to initiate the blood pressure measurement based on an instruction input by the subject to initiate the blood pressure measurement or based on a trigger signal autonomously generated by the blood pressure monitor 1, as described above. The blood pressure monitor 1 may be a blood pressure monitor using a blood pressure detection method of a continuous measurement type such as a pulse transmit time (PTT), tonometry, optical, radio wave, or ultrasonic wave method. The PTT method is a method of measuring the pulse transmit time (PTT) and estimating a blood pressure value from the measured pulse transmit time. The tonometry method includes a method of measuring the blood pressure value based on information detected by a pressure sensor by bringing the pressure sensor into direct contact with a measurement area through which the artery runs, such as the radial artery of the wrist. The optical, radio wave, or ultrasonic wave method is a method of measuring the blood pressure value based on a reflection wave obtained by applying light, radio waves, or ultrasonic waves on the blood vessel.

The blood pressure monitor of a continuous measurement type can determine whether or not the subject is wearing the blood pressure monitor at the set time point, in accordance with the presence or absence of a signal from the sensor arranged to implement any one of the above described various methods.

The functions described in the above embodiments may be realized by adopting a circuit. The circuit may be a circuit dedicated to realizing a particular function, or may be a general-purpose circuit such as a processor.

At least part of the processing of the above-mentioned embodiment can be realized by adopting a general-purpose computer as the basic hardware. The program that realizes the above processing may be provided in a computer-readable storage medium. The program is stored as a file of an installable format or a file of an executable format in a storage medium. The storage medium should be sufficient if it can store programs and is readable by the computer. The storage medium may include a magnetic disk, an optical disk (e.g., a compact disk read only memory (CD-ROM), a compact disk recordable (CD-R), a digital versatile disk (DVD)), a magneto-optical disk (MO), and a semiconductor memory. The program for realizing the above processing may be stored on a computer (server) connected to a network such as the Internet, and downloaded to a computer (client) via the network.

In the above embodiment, the blood pressure measurement has been explained. The embodiment is not limited thereto but is also applicable to the measurement of other biological information including the amount of activity, number of steps walked, electrocardiographs, pulse rate, and body temperature.

In other words, the present invention is not limited to the above-described embodiment as-is, and can be embodied by modifying the components without departing from the scope of the invention at the implementation stage. Furthermore, various inventions can be produced by suitably combining components disclosed in the embodiment. For example, some components may be deleted from all the components shown in the embodiment. In addition, the components over different embodiments may be appropriately combined.

Further, part or all of the above-described embodiment can be described as in the following additional notes, but is not limited thereto.

### Additional Note 1

A biological information measurement apparatus, including:

a processor configured to:
acquire information indicating a set time point being set to determine whether or not a subject is wearing the biological information measurement apparatus;
determine whether or not the subject is wearing the biological information measurement apparatus at the set time point; and
if it is determined that the subject is not wearing the biological information measurement apparatus at the set time point, output an instruction signal that instructs provision of assistance to the subject in wearing the biological information measurement apparatus; and

a memory configured to store instructions to operate the processor.

### Additional Note 2

A wearing assist method comprising:

acquiring, by at least one processor, information indicating a set time point being set to determine whether or not a subject is wearing a biological information measurement apparatus;

determining, by the at least one processor, whether or not the subject is wearing the biological information measurement apparatus at the set time point; and

if it is determined that the subject is not wearing the biological information measurement apparatus at the set time point, outputting, by the at least one processor, an instruction signal that instructs provision of assistance to the subject in wearing the biological information measurement apparatus.

### REFERENCE SIGNS LIST

1 Blood pressure monitor
10 Main body
10A Casing
10B Watch glass
10C Back cover
20 Belt
30 Cuff structure
30a One end
30b Other end
30c Inner surface
70 Server
80 Mobile terminal
90 Left wrist
91 Radial artery
92 Ulnar artery
93 Radius
94 Ulna
95 Tendon
101 Display unit
102 Operation unit
103 CPU
103A Set time information acquisition unit
103B Determination unit
103C Blood pressure measuring unit
103D Current location information acquisition unit
103E Priority information acquisition unit
103F Schedule information acquisition unit
103G Instruction signal output unit
104 Memory
105 Acceleration sensor
106 Temperature and humidity sensor
107 Atmospheric sensor
108 Communication unit
109 GPS receiver
110 Battery
111 First pressure sensor
112 Second pressure sensor
113 Pump driving circuit
114 Pump
115 On/off valve
116 Sound output unit
201 First belt unit
201a Proximal portion
201b Distal portion
202 Second belt unit
202a Proximal portion
202b Distal portion
202c Small openings
203 Buckle
203A Frame
203B Bar
203C Connecting rod
204 Belt holder
301 Curler
302 Pressing cuff
303 Back plate
304 Sensing cuff
304A First sheet
304B Second sheet
401 Connecting rod
402 Connecting rod
501 Flexible tube
502 Flexible tube
503 First channel member
504 Second channel member

1. A biological information measurement apparatus comprising:
a processor configured to:
acquire information indicating a set time point being set to determine whether or not a subject is wearing the biological information measurement apparatus;
determine whether or not the subject is wearing the biological information measurement apparatus at the set time point;
acquire information indicating a priority level associated with the set time point; and
if it is determined that the subject is not wearing the biological information measurement apparatus at the set time point and when the priority level indicated by the acquired information is a first priority level, output an instruction signal instructing provision of assistance to the subject in wearing the biological information measurement apparatus, and when the priority level indicated by the acquired information is a second priority level being different from the first priority level, not to output the instruction signal.

2. The biological information measurement apparatus according to claim 1, wherein
the set time point is a time point for performing measurement of biological information or a time point a predetermined time length prior to the time point for performing the measurement of the biological information, and
the set time point is based on a time point entered through a user interface for entering the time point for performing the measurement of the biological information.

3. The biological information measurement apparatus according to claim 1, wherein
the set time point is a time point scheduled for the subject to leave a predetermined location or a time point a predetermined time length prior to the time point scheduled for the subject to leave the predetermined location, and
the set time point is based on a time point entered through a user interface for entering the time point scheduled for the subject to leave the predetermined location.

4. The biological information measurement apparatus according to claim 1, wherein the processor is further configured to:
acquire information indicating a current location of the biological information measurement apparatus; and
change a type of the assistance in accordance with the current location.

5. The biological information measurement apparatus according to claim 1, wherein the processor is further configured to:
acquire information indicating a schedule of the subject; and
set a time point for providing the, assistance in accordance with the schedule.

6. A wearing assist method comprising:
acquiring information indicating a set time point being set to determine whether or not a subject is wearing a biological information measurement apparatus;
determining whether or not the subject is wearing the biological information measurement apparatus at the set time point;
acquiring information indicating a priority level associated with the set time point; and
if it is determined that the subject is not wearing the biological information measurement apparatus at the set time point and when the priority level indicated by the acquired information is a first priority level, outputting an instruction signal instructing provision of assistance to the subject in wearing the biological information measurement apparatus, and when the priority level indicated by the acquired information is a second priority level being different from the first priority level, not outputting the instruction signal.

7. A non-transitory computer readable medium including computer executable instructions, wherein the instructions, when executed by a processor, cause the processor to perform a method comprising:
acquiring information indicating a set time point being set to determine whether or not a subject is wearing a biological information measurement apparatus;
determining whether or not the subject is wearing the biological information measurement apparatus at the set time point;
acquiring information indicating a priority level associated with the set time point; and
if it is determined that the subject is not wearing the biological information measurement apparatus at the set time point and when the priority level indicated by the acquired information is a first priority level, outputting an instruction signal instructing provision of assistance to the subject in wearing the biological information measurement apparatus, and when the priority level indicated by the acquired information is a second priority level being different from the first priority level, not outputting the instruction signal.
